(19) United States
(12) Patent Application Publication
(10) Pub. No.: US 2004/0143208 A1
(43) Pub. Date: Jul. 22, 2004

(54) Irradiation chamber

(76) Inventor: Stephen Gara, Collegeville, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/742,343

(22) Filed: Dec. 19, 2003

Related U.S. Application Data
(60) Provisional application No. 60/434,961, filed on Dec. 20, 2002.

(51) Int. Cl. A61M 37/00

(52) U.S. Cl. 604/6.01; 604/6.08

(57) ABSTRACT

The present invention provides an irradiation chamber comprising: a rigid first plate having a first surface and a second surface having a raised boundary surrounding a plurality of raised partitions; a rigid second plate having a first surface and a second surface having a raised boundary surrounding a plurality of raised partitions; wherein the second surface of said rigid first plate is contacted with second surface of said rigid second plate thereby forming a chamber; said chamber, defined by the raised boundary surrounding the plurality of raised partitions which extend from said second surface of said first plate and said second surface of said second plate, said chamber having a first port and a second port, wherein a plurality of channels are formed by said partition and are in fluid communication with the first port and second port. The present invention also provides a method of using the irradiation chamber in the treatment of cells.

### FIELD OF THE INVENTION

This invention relates to a chamber for holding biological fluid or components thereof such as blood or blood products to facilitate their exposure to electromagnetic radiation such as UV light. This invention also relates to a method of treating cells with radiation thereby inducing apoptosis of the cells such as for the extracorporeal treatment of blood cells, especially leukocytes, with UV radiation.

### BACKGROUND OF THE INVENTION

A number of human diseases are mediated by the overproduction of certain types of leukocytes such as lymphocytes. Excessive or abnormal lymphocyte populations result in numerous adverse effects to patients including the functional impairment of bodily organs, leukocyte mediated autoimmune diseases and leukemia related disorders. Photopheresis therapy has been employed to treat conditions such as CLL, Scleroderma, SLE, Psoriasis, Pemphigus, Psoriatic Arthritis, Atopic dermatitis, ATL, AIDS (ARC), Rheumatoid Arthritis, MS, and organ transplant rejections.

U.S. Pat. Nos. 4,321,919; 4,398,906; 4,428,744; and 4,464,166 to Edelson describe methods for treating blood. These patents describe methods for treating component or components of blood which in turn ameliorate, reduce the severity, or provide relief from the conditions for patients whose blood is treated. In general, the methods comprise treating the blood with a dissolved photoactivatable drug, such as psoralen, which is capable of forming photoadducts with DNA in the presence of U.V. radiation. It is believed that covalent bonding results between the psoralen and the lymphocyte nucleic acid thereby effecting metabolic inhibition of the thusly treated cells. Following extracorporeal radiation, the cells are returned to the patient where they are thought to be cleared by natural processes but at an accelerated pace believed attributable to disruption of membrane integrity, alteration of DNA within the cell, or the like conditions often associated with substantial loss of cellular effectiveness or viability.

In a photophoresis treatment, one or several components of a patient's blood is exposed to UV radiation in the presence of a photoactivable compound. The phases of a photopheresis treatment comprises collection of the leukocyte-enriched blood or buffy coat, photoactivation and reinfusion with the aid of a patient treatment instrument. Example of an instrument which performs a photopheresis procedure is shown in “The UVAR XTS System: Engineering That Reflects Innovation”, dated March 1998. Therakos, Inc. The collection of the buffy coat volume as well as the number of cycles are predetermined by a physician. Assume, that the predetermined volume and cycle conditions are set as follows: 350 mls of plasma, 250 mls of buffy coat, and 5 cycles. In each cycle, the apparatus will collect 250/5 or 50 mls of buffy coat before ending the cycle and thereupon emptying the centrifuge bowl and returning all nonleukocyte fluids, predominantly erythrocytes and perhaps excess plasma, to the patient. Prior to the collection of the 50 mls, plasma will emerge from the centrifuge and will be collected either until the full 350 mls are collected or, until the buffy coat emerges.

More specifically, the instrument collects and separates blood on a continuous basis as it is withdrawn from the patient and returns untreated portions to the patient while concurrently irradiating the buffy coat in the irradiation chamber with UV light. The irradiation, preferably UV light, photoactivates the photoactivatable agent in contact with the desired blood portion while the agent and the cells (or other patient fluid) is contained within the flat plate irradiation chamber. Following photoactivation, the treated cells will be returned to the patient utilizing a drip chamber gravity feed infusion line incorporated in a photopherisis blood tubing set. The photopheresis blood tubing set has multiple lines used for collecting, photoactivating and reinfusing the leukocyte-enriched blood. These lines are the patient/heparin line, the collection/return line, the bowl outlet line and the photoactivation line.

The instrument also controls blood and recirculation pump speed/direction and also supplies power to the centrifuge. A microprocessor and discrete logic circuits monitor operating parameters throughout treatment and display instrument status and conditions. Microprocessor controls assist the operator in the various stages of the photopheresis procedure.

The irradiation chamber has a thin sterile fluid pathway constructed of UVA-transparent acrylic. The irradiation chamber's design allows it to be inserted between the two banks of UVA lamps for photoactivation. Suitable sources for UV lamps include the Sylvania FR15“T8/350BL/HO/180 degree with 2011 phosphorus bulb which is in the so-called fluorescent tube form.

Photoactivable compounds that can be used with the present invention include, but are not limited to, primaryamino-pyrone-linked and benzene-linked psoralens disclosed in U.S. Pat. No. 6,455,286. Compound 8-methoxy psoralen is most preferred among photoactivatable compounds in the psoralen class.

There are many patents and publications that disclose containers/chambers for treatment of fluid, including blood products, by irradiation (e.g. U.S. Pat. Nos. 3,628,445; 4,708,715; 4,737,140; D298,279; 4,866,282; 4,876,014; 4,897,789; 4,915,683; 5,039,483; 5,304,113; 5,290,221; 5,868,695; 5,951,509; 6,133,566; 6,312,593 and US2001/0024623).

One device, is shown in publications WO 98/22165, WO 98/22163 and in a brochure dated March 1998 by Therakos, Inc., “The UVAR XTS System: Engineering That Reflects Innovation.” These publications show an irradiation chamber having seven channels where blood products pass through while being irradiated with UV light. The irradiation chamber, also known as the PHOTOCEPTOR® Photoactivation Chamber, is made from two differently shaped plates. One side of the irradiation chamber has both input and output ports protruding above the surface of the plate (FIG. 1 shows input and output ports 770 and 780 on opposite side of front plate) while the other side is essentially flat (FIG. 1, front plate shown). Furthermore, one plate has partitions extending from its surface, which are then sealed to recesses of the other plate to form a serpentine pathway.

The current manufacturing process to produce the PHOTOCEPTOR® Photoactivation Chamber results in a number of rejected products. This is due to uneven application of RF energy between the two different plates in the manufacturing process: one plate has partitions extending perpendicular from its surface and one has recesses on its surface to receive the partitions. In addition to the difficulty in the production of this irradiation chamber, the manufacturing process requires two injection mold tools for the two plates.

Displacement of a desired buffy coat from the PHOTOCEPTOR® Photoactivation Chamber requires introduction of another fluid such as plasma or saline because the design of the irradiation chamber does not effectively utilize gravity to transfer blood components. The volume of such fluid usually exceeds the volume of the irradiation chamber due to a wash volume required for residual buffy. In a typical photopherisis treatment for normal adult patients requiring several cycles of separation, irradiation, and reinfusion of blood, additional volumes of fluid may be returned to the patient. However in a small patient (such as children) or a patient whose vascular system is easily overloaded with fluids, the extra volume of fluid provided in extracorporeal photopherisis potentially presents a problem.

The design of the irradiation chamber of the present invention allows for gravity-assisted transferring of a desired fluid. Gravity-assisted transferring of fluid results in greater efficiency in fluid transfer management and allows for reducing the total volume being processed in an extracorporeal photopheresis treatment. For example, if the desired fluid is bufffy coat, then displacement of the buffy coat is accomplished by a displacing plasma and/or saline with the aid of gravity. A washing volume may be needed for residual buffy coat, however this washing volume is small relative to that needed for the PHOTOCEPTOR® Photoactivation Chamber. This ability to have a reduced processed volume is desirable for small patients or those with compromised vascular systems.

The present invention also allows for efficient manufacturing of irradiation chamber due to fewer mold tools needed because the two plates forming the irradiation chamber are identical. Only one mold is required for the production of the irradiation of the present invention. Furthermore, the chambers giving rise to the serpentine pathway of the irradiation chamber are formed by RF welding of partitions extending perpendicular from a plate's surface. RF welding of partitions of the present invention to form a serpentine pathway provides fewer rejected product due to even application of RF energy to partitions of plates.

It is a further related object of this invention to provide an irradiation chamber that can be used with a continuous on-line patient treatment system wherein collection, separation, and cell treatment occur simultaneously.

### BRIEF DESCRIPTION OF THE DRAWINGS

These and still other objects of the invention will become apparent upon study of the accompanying drawings:

FIG. 1 shows a front view of a PHOTOCEPTOR® Photoactivation Chamber.

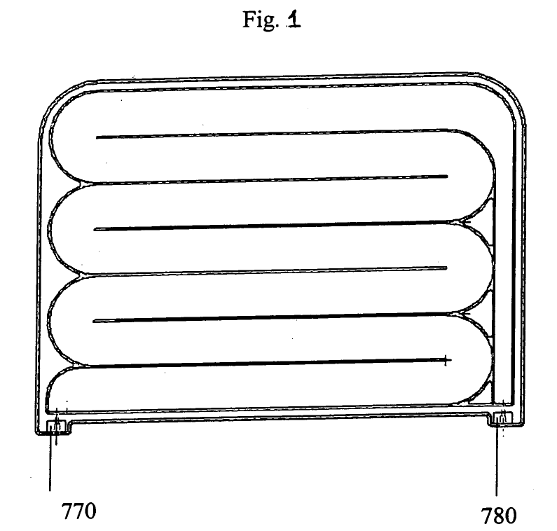

FIG. 2 shows a front view of the claimed irradiation chamber.

FIG. 3 shows a side longitudinal view of the claimed irradiation chamber.

FIG. 4 shows a side transverse view of the claimed irradiation chamber.

FIG. 5 shows a cut-away view of a section of the first plate and the second plate prior to being joined together.

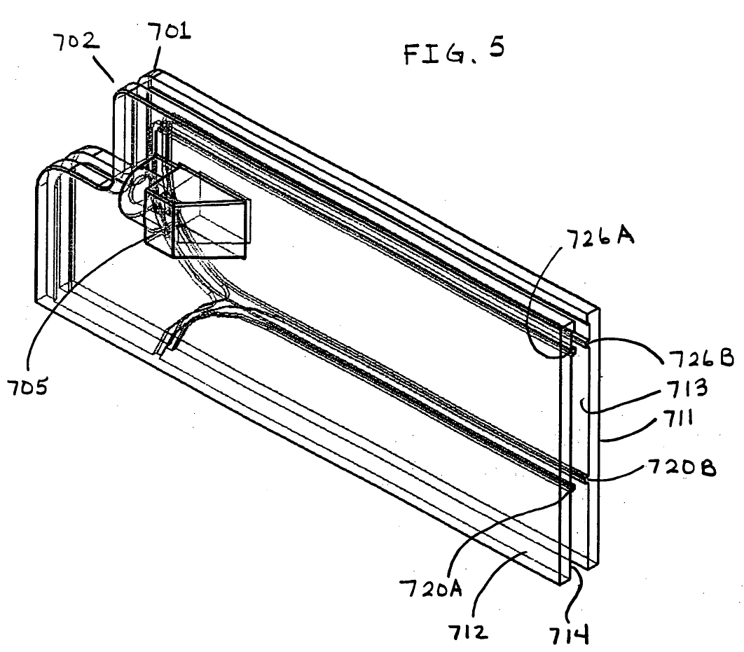

FIG. 6 shows a cut-away dimensional end view of an irradiation chamber.

FIG. 7 shows an irradiation chamber in a UVA light assembly.

### SUMMARY OF THE INVENTION

The present invention provides for an irradiation chamber comprising: a rigid first plate having a first surface and a second surface having a raised boundary surrounding a plurality of raised partitions; a rigid second plate having a first surface and a second surface having a raised boundary surrounding a plurality of raised partitions; wherein the second surface of said rigid first plate is contacted with second surface of said rigid second plate thereby forming a chamber; said chamber, defined by the raised boundary surrounding the plurality of raised partitions which extend from said second surface of said first plate and said second surface of said second plate, said chamber having a first port and a second port, wherein a plurality of channels are formed by said partition and are in fluid communication with the first port and second port.

In accordance with the principles and objects of the present invention there is provided a method of use for the irradiation chamber in the patient treatment instrument which provides for an treating cells, particularly derived from blood, comprising collecting cells in an aqueous media from a patient; and irradiating the desired cells to induce apoptosis.

### DETAILED DESCRIPTION

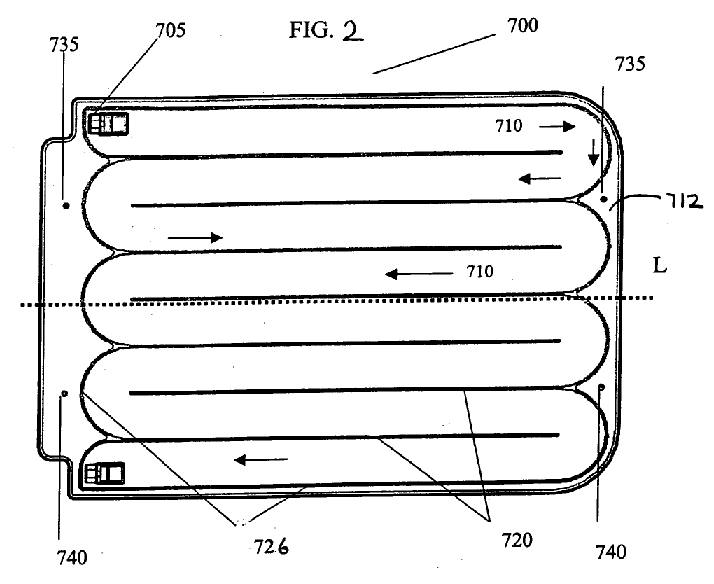

Irradiation chamber 700 (FIG. 2) is formed by joining two plates, a front and a back plate having a thickness of preferably about 0.06 in. to about 0.2 in., which are preferably comprised of a material ideally transparent to the wavelength of electromagnetic radiation. In the case of ultraviolet A radiation, polycarbonate has been found most preferred although other materials such as acrylic may be employed. Similarly, many known methods of bonding may be employed and need not be expanded on here.

The first plate 702 has a first surface 712 and a second surface 714. In a preferred embodiment the first plate 702 has a first port 705 on a first surface 712, in fluid communications with the second surface 714. The second surface 714 of the first plate 702 has a raised boundary 726A defining an enclosure. The boundary 726A preferably extends substantially perpendicular from the second surface 714 (i.e. about 80-100 degrees). Extending from the second surface 714 (preferably substantially perpendicularly) are raised partitions 720A. The boundary 726A surrounds the partitions 720A. One end of each partition 720A extends and contacts the boundary 726A.

The second plate 701 has a first surface 711 and a second surface 713. In a preferred embodiment the second plate 701 preferably has a second port 730 on a first surface 711, in fluid communications with the second surface 713. The second surface 713 of the back plate 701 has a raised boundary 726B defining an enclosure. The boundary 726B preferably extends substantially perpendicular from the second surface 713 (i.e. about 80-100 degrees). Extending from the second surface 713 (preferably substantially perpendicular) are raised partitions (720B). The boundary 726B surrounds the partitions 720B. One end of each partition 720A extends and contacts one side of boundary (726B).

The joining of the second surfaces of the first and second plates results in a fluid tight junction between boundaries 726A and 726B thereby forming boundary 726. Partitions 720A and 720B are also joined forming a fluid tight junction thereby forming partition 720. The boundary 726 forms an irradiation chamber 700 and together with the partitions 720 provides a pathway 710 having channels 715 for conducting fluid. The pathway maybe serpentine, zig-zag, or dove-tailed. Currently preferred is a serpentine pathway.

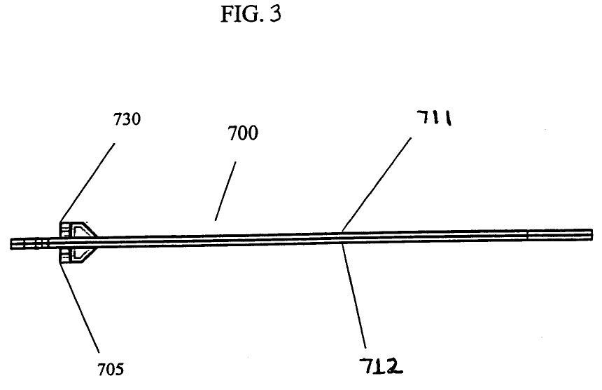

With reference to FIGS. 2 and 3, irradiation chamber 700 comprises a serpentine pathway 710 for conducting patient fluid from inlet port 705 to outlet port 730, i.e., the serpentine pathway 710 is in fluid communication with inlet port 705 of front plate 702 and outlet port 730 of back plate 701. Self-shielding effects of the cells is reduced while the cells are photoactivated by irradiation impinging upon both sides of irradiation chamber 700.

FIG. 2 shows pin 740 and recess 735 which align the two plates of irradiation chamber prior to being joined together in a sealing arrangement by RF welding, heat impulse welding, solvent welding or adhesive bonding. Joining of the plates by adhesive bonding and RF welding is more preferred. Joining of the front and back plates by RF welding is most preferred as the design of the raised partitions 720 and perimeter 725 minimizes flashing and allows for even application of RF energy. Locations of pin 740 and recess 735 may be inside serpentine pathway 710 or outside of serpentine pathway 710 (as shown in FIG. 2). FIG. 2 also shows a view of an irradiation chamber with axis L. Rotation of chamber 180 degree about axis L gives the original configuration of the irradiation chamber. The irradiation chamber of the present invention has C₂ symmetry about axis L.

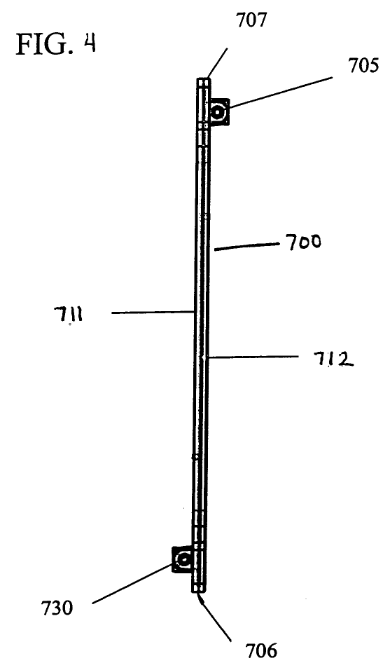

Referring to FIGS. 2, 4, and 7, the leukocyte enriched blood, plasma, and priming solution are delivered through inlet port 705 of front plate 702 of irradiation chamber 700 into channel 715. The channel 715 in the irradiation chamber 700 is relatively “thin” (e.g. on the order of approximately 0.04″ as distance between two plates) in order to present large surface area of leukocyte rich blood to irradiation and reduce the self-shielding effects encountered with lower surface area/volume ratios. The cross section shape of channel 715 is substantially rectangular (e.g. rectangular, rhomboidal or trapezoidal) which has as its long side the distance between partition 720 and the distance between the plates as its short side. The shape of the cross section is designed for optimal irradiation of cells passing through channel 715. While a serpentine pathway 710 is preferred in order to avoid or minimize stagnant areas of flow, other arrangements are contemplated.

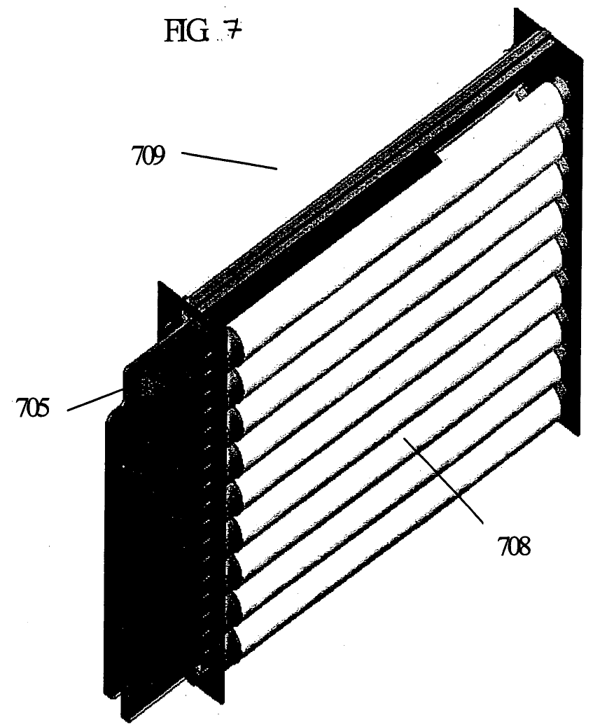

The irradiation chamber 700 allows efficient activation of photoactivatable agents by irradiation from a light array assembly such as the PHOTOSETTE®'s two banks of UVA lamps (708) for activation (FIG. 7). The irradiation chamber and UVA light assembly (709) is designed to be used in a setting where edge 706 is oriented downward and edge 707 points upward. In this orientation, fluids entering input port 705 can exit from outlet port 730 with the aid of gravity. In the most preferred embodiment, irradiation of both sides of the irradiation chamber takes place concurrently while still permitting facile removal of the chamber.

The irradiation chamber's fluid pathway loops to form two or more channels in which the leukocyte-enriched blood is circulated during photoactivation by UVA light. Preferably, irradiation chamber has between 4 to 12 channels. More preferably, the irradiation chamber has 6 to 8 channels. Most preferably, the irradiation chamber has 8 channels.

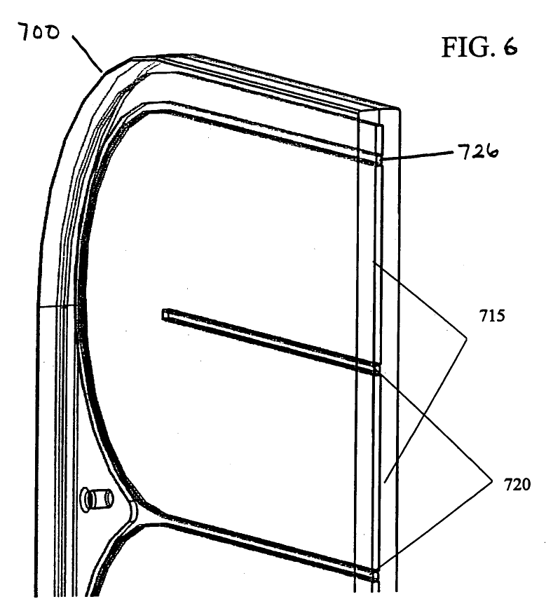

FIG. 6 shows cut-away views of the irradiation chamber. The channels 715 of serpentine pathway 710 are formed by the joining of raised partition 720 and perimeter 726 of the plates.

The irradiation chamber of the present invention can be made from a biocompatible material and can be sterilized by known methods such as heating, radiation exposure or treatment with ethylene oxide (ETO).

In another embodiment of the present invention a method is provided for irradiating cells using the claimed irradiation chamber during extracorporeal treatment of cells with electromagnetic radiation (UVA) to be used in the treatment of a patient (such as to induce apoptosis in the cells and administer the cells into the patient). Preferably the cells treated will be white cells.

In one embodiment of this method a photoactivatable or photosensitive compound is first administered to at least a portion of the blood of a recipient prior to the extracorporeal treatment of the cells. The photoactivatable or photosensitive compound may be administered in vivo (e.g., orally or intravenously). The photosensitive compound, when administered in vivo may be administered orally, but also may be administered intravenously and/or by other conventional administration routes. The oral dosage of the photosensitive compound may be in the range of about 0.3 to about 0.7 mg/kg., more specifically, about 0.6 mg/kg.

When administered orally, the photosensitive compound may be administered at least about one hour prior to the photopheresis treatment and no more than about three hours prior to the photopheresis treatment. If administered intravenously, the times would be shorter.

Alternatively, the photosensitive compound may be administered prior to or contemporaneously with exposure to ultraviolet light. The photosensitive compound may be administered to whole blood or a fraction thereof provided that the target blood cells or blood components receive the photosensitive compound. A portion of the blood could first be processed using known methods to substantially remove the erythrocytes and the photoactive compound may then be administered to the resulting enriched leukocyte fraction. In one embodiment, the blood cells comprise white blood cells, specifically, T-cells.

In accordance with the present invention, the photoactivatable or photosensitive compound may, in the case of some psoralens, be capable of binding to nucleic acids upon activation by exposure to electromagnetic radiation of a prescribed spectrum, e.g., ultraviolet light.

Photoactive compounds for use in accordance with the present invention may include, but are not limited to, compounds known as psoralens (or furocoumarins) as well as psoralen derivatives such as those described in, for example, U.S. Pat. No. 4,321,919 and U.S. Pat. No. 5,399,719. The photoactivatable or photosensitive compounds that may be used in accordance with the present invention include, but are not limited to, psoralen and psoralen derivatives; 8-methoxypsoralen; 4,5′8-trimethylpsoralen; 5-methoxypsoralen; 4-methylpsoralen; 4,4-dimethylpsoralen; 4-5′-dimethylpsoralen; 4′-aminomethyl-4,5′,8-trimethylpsoralen; 4′-hydroxymethyl-4,5′,8-trimethylpsoralen; 4′,8-methoxypsoralen; and a 4′-(omega-amino-2-oxa) alkyl-4,5′,8-trimethylpsoralen, including but not limited to 4′-(4-amino-2-oxa)butyl-4,5′,8-trimethylpsoralen. In one embodiment, the photosensitive compound that may be used comprises the psoralen derivative, amotosalen (S-59) (Cerus, Corp., Concord, Calif.). See, e.g., U.S. Pat. Nos. 6,552,286; 6,469,052; and 6,420,570. In another embodiment, the photosensitive compound that may be used in accordance with the invention comprises 8-methoxypsoralen.

Methoxsalen is a naturally occurring photoactive substance found in the seed of the Ammi majus (umbelliferae plant). It belongs to a class of compounds known as psoralens or furocoumarins. The chemical name is 9-methoxy-7H-furo[3,2-g][1]-benzopyran-7-one. The formulation of the drug is a sterile liquid at a concentration of 20 mcg/mL in a 10 mL vial. See http://www.therakos.com/TherakosUS/pdf/uvadexpi.pdf. Toxicology studies of extracorporeal photopheresis and different dosages of UVADEX® and ultraviolet light in beagle dogs is located in the investigator's brochure.

Next, the portion of the subject's blood, recipient's blood, or the donor's blood to which the photoactive compound has been administered is treated by subjecting the portion of the blood to photopheresis using ultraviolet light. The photopheresis treatment in the treatment methods according to the present invention may be carried out using long wavelength ultraviolet light (UVA) at a wavelength within the range of 320 to 400 μm. Such a range is not limiting, however, but is merely provided as an example. The exposure to ultraviolet light during the photopheresis treatment may have a duration of sufficient length to deliver, for example, about 1-2 J/cm² to the blood.

The photopheresis step is carried out in vitro using an extracorporeal photopheresis apparatus. An extracorporeal photopheresis apparatus that may be used in the methods according to the invention is currently manufactured by Therakos, Inc., (Exton, Pa.) under the name UVAR®. A description of such an apparatus may be found, for example, in U.S. Pat. No. 4,683,889.

In one embodiment, when the photopheresis step is carried out in vitro, at least a fraction of the treated blood is returned to the subject, recipient, or donor. The treated blood or the treated enriched leukocyte fraction (as the case may be) may then be administered back to the subject, recipient, or donor. Alternatively, the blood may be separated on a standard apheresis-type device and photoactivated on a separate device.

A specific but non-limiting example of a photopheresis system is the UVAR® System, which uses a photospheres treatment system and consists of three phases including: 1) the collection of a buffy-coat fraction (leukocyte-enriched), 2) irradiation of the collected buffy coat fraction, and 3) reinfusion of the treated white blood cells. The collection phase has six cycles of blood withdrawal, centrifugation, and reinfusion steps. During each cycle, whole blood is centrifuged and separated in a pediatric pheresis bowl. From this separation, plasma (volume in each cycle is determined by the UVAR®. Instrument operator) and 40 ml buffy coat are saved in each collection cycle. The red cells and all additional plasma are reinfused to the patient before beginning the next collection cycle. Finally, a total of 240 ml of buffy coat and 300 ml of plasma are separated and saved for UVA irradiation.

The irradiation of the leukocyte-enriched blood within the irradiation circuit begins during the buffy coat collection of the first collection cycle. The collected plasma and buffy coat are mixed with 200 ml of heparinized normal saline and 200 mg of UVADEX®. (water soluble 8-methoxypsoralin). This mixture flows in a 1.4 mm thick layer through the irradiation chamber of the present invention. The irradiation chamber 700, is inserted between two banks of UVA lamps of the PHOTOSETTE® (see FIG. 7). PHOTOSETTE® UVA lamps irradiate both sides of this UVA-transparent irradiation chamber, permitting a 180-minute exposure to ultraviolet A light, yielding an average exposure per lymphocyte of 1-2 J/cm². The final buffy coat preparation contains an estimated 20% to 25% of the total peripheral blood mononuclear cell component and has a hematocrit from 2.5% to 7%. Following the photoactivation period, the cells are removed from the irradiation chamber.

In a preferred embodiment of the present invention the cells are removed by the action of gravity and any cells remaining in the chamber are displaced from the chamber with additional fluid selected from the group consisting of saline, plasma and combinations thereof. For patients who are small such as children (e.g. under 30 kg) or patients whose vascular system is easily overloaded with fluids the amount of additional fluid used in the irradiation chamber will preferably be not more than 2× the volume of the chamber, preferably not more than 1× the volume of the chamber, more preferably not more than 0.5× the volume of the chamber 0.25× the volume of the chamber. The treated cells volume is reinfused to the patient preferably over a 30 to 45 minute period.

For a description of similar photopheresis systems useful in the methods of the present invention, see U.S. patent application Ser. No. 09/480,893, which is expressly incorporated herein by reference. Also useful herein are the methods and systems described in U.S. Pat. Nos. 5,951,509; 5,985,914; 5,984,887, 4,464,166; 4,428,744; 4,398,906; 4,321,919; PCT Publication Nos. WO 97/36634; and WO 97/36581, all of which are entirely expressly incorporated herein by reference.

Another system that may be useful in the methods of the present invention is described in U.S. patent application Ser. No. 09/556,832, which is entirely expressly incorporated herein by reference. The system described therein relates to systems and apparatus by which the net fluid volume collected or removed from a patient may be reduced during a medical treatment process such as ECP. By way of example, an ECP process such as the UVAR® process (Therakos, Inc., Exton, Pa.) removes blood from a patient, separates the buffy coat from the plasma and red blood cells and replaces the biological fluids in a batch process. When blood is removed from the patient, however, a volume deficit is created within the patient. This volume deficit is particularly detrimental in small children and the elderly or in patients that suffer from certain illnesses or diseases because their blood has a higher percentage of plasma relative to the cellular components. This volume imbalance requires that a greater volume of blood be drawn from the patient to obtain the required amount of red blood cells. This especially impacts infants and sick children who may have low body weight and hemocrit percentages of 25-30% which is significantly lower than the normal average of 45%. The need thus arose to be able to detect small incremental changes in natural fluid ratios within the body and to use these measurements to create a process by which the net fluid volume collected or removed from a patient may be reduced during a medical treatment process.

The effective amount of light energy that is delivered to the biological fluids may be determined using the methods and systems described in U.S. Pat. No. 6,219,584, which is entirely expressly incorporated herein by reference. Indeed, the application of ECP to the various diseases described herein may require an adjustment of the amount of light energy to optimize the treatment process.

Furthermore, the photosensitizing agent used in the ECP process may be removed prior to returning the treated biological fluid to the patient. For example, the UVAR® System utilizes Methoxsalen (UVADEX®) in the ECP process. Methoxsalen belong to a group of compounds known as psoralens. The exposure to methoxsalen or other psoralens may cause undesirable effects on the subject, recipient, or donor such as phototoxicity or other toxic effects associated with psoralen and their decomposition products. Therefore, the psoralen, psoralen derivatives, or psoralen decomposition products that may remain in the biological fluid may be removed after UV exposure. A process for the removal of psoralen biological fluids is described in U.S. Pat. No. 6,228,995, which is entirely expressly incorporated herein by reference.

The ECP system useful in the methods of the present invention may incorporate one or more components described in U.S. Pat. No. 6,069,687 (contaminant detector), U.S. Pat. No. 5,921,951 (steady flow rate pump), U.S. Pat. No. 5,569,928 (photoactivation light array), U.S. Pat. No. 5,459,322 (ultraviolet light chamber), U.S. Pat. No. 5,330,420 (hemolysis detector), U.S. Pat. No. 5,308,309 (securing system for centrifuge chamber), U.S. Pat. No. 4,921,473 (multicomponent fluid separation and irradiation system); and U.S. application Ser. No. 09/389,463 (uninterrupted flow pump apparatus), all of which are entirely expressly incorporated herein by reference.

Upon study of the accompanying figures, and the foregoing description, it will become readily apparent to the skilled artisan that numerous alternatives may be made to the foregoing inventions without departing from either the spirit or scope of the instant invention.

What is claimed is:

1. An irradiation chamber comprising:
a rigid first plate having a first surface and a second surface said second surface having a raised boundary surrounding a plurality of raised partitions;
a rigid second plate having a first surface and a second surface, said second surface having a raised boundary surrounding a plurality of raised partitions;
wherein the second surface of said rigid first plate is contacted with second surface of said rigid second plate thereby forming a chamber; said chamber, defined by the raised boundary surrounding the plurality of raised partitions which extend from said second surface of said first plate and said second surface of said second plate, said chamber having a first port and a second port, wherein a plurality of channels are formed by said partition providing fluid communication with the first port and second port.

2. The irradiation chamber of claim 1 wherein said partitions are essentially evenly spaced apart from each other.

3. The irradiation chamber of claim 2 wherein the number of partitions ranges from three to eleven.

4. The irradiation chamber of claim 3 wherein the number of partitions is seven.

5. The irradiation chamber of claim 2 wherein the channels form a serpentine fluid pathway providing fluid communications between said first and second port.

6. The irradiation chamber of claim 5 wherein the first port is on the first surface of the first plate.

7. The irradiation chamber of claim 6 wherein the second port is on the first surface of the second plate.

8. The irradiation chamber of claim 7, wherein the channel has a thickness of about 0.04 inches.

9. The irradiation chamber of claim 1, wherein the rigid first plate and second plate are made of a material that does not substantially absorb UV radiation having wavelength in the range of 180 to 420 nm.

10. The irradiation chamber of claim 9, wherein the rigid first plate and second dplate are made of material selected from the group consisting of polycarbonate and acrylic.

11. An irradiation chamber of claim 1 wherein said rigid first plate is identical to said rigid second plate.

12. A method for forming irradiated blood components comprising:
(a) collecting blood from a patient;
(b) separating a desired component from said blood;
(c) irradiating the desired component to form an irradiated blood product in an irradiation chamber comprising:
a rigid first plate having a first surface and a second surface said second surface having a raised boundary surrounding a plurality of raised partitions;
a rigid second plate having a first surface and a second surface, said second surface having a raised boundary surrounding a plurality of raised partitions;
wherein the second surface of said rigid first plate is contacted with second surface of said rigid second plate thereby forming a chamber; said chamber, defined by the raised boundary surrounding the plurality of raised partitions which extend from said second surface of said first plate and said second surface of said second plate, said chamber having a firs port and a second port, wherein a plurality of channels are formed by said partition providing fluid communication with the first port and second port; and
(d) collecting said irradiated blod product.

13. The method of claim 12 wherein the irradiated blood product contains cells that have been induced to undergo apoptosis by said irradiation.

14. The method of claim 12 further comprising contacting the desired component with a photoactivable agent and irradiating the desired component prior to irradiating said desired component to form an irradiated blood product.

15. The method of claim 12 further comprising returning the irradiated blood product to the patient.

16. The method of claim 12 further comprising washing the irradiation chamber with less than 1× volume of a fluid selected from the group consisting of saline, plasma or combinations thereof to displace any irradiated blood product remaining in the irradiation chamber thereby forming a wash solution.

17. The method of claim 15 wherein the was solution is returned to the patient.

18. A method of treating a patient comprising
(a) collecting blood from a patient;
(b) separating a desired component from said blood on a continuous basis;
(c) contacting the desired component with a photoactivable agent;
(d) irradiating the desired component and the photoactivable agent to form an irradiated blood product in an irradiation chamber comprising:
a rigid first plate having a first surface and a second surface said second surface having a raised boundary surrounding a plurality of raised partitions;
a rigid second plate having a first surface and a second surface, said second surface having a raised boundary surrounding a plurality of raised partitions
wherein the second surface of said rigid first plate is contacted with second surface of said rigid second plate thereby forming a chamber; said chamber, defined by the raised boundary surrounding the plurality of raised partitions which extend from said second surface of said first plate and said second surface of said second plate, said chamber having a first port and a second port, wherein a plurality of channels are formed by said partition providing fluid communication with the first port and second port; and
(e) returning the irradiated blood product to the patient.

19. The method of claim 17, wherein the desired compnent is buffy coat.

20. The method of claim 17, wherein the photoactivable agent is 8-MOP.

21. The method of claim 17 wherein the desired component is buffy coat and the photoactivable agent is 8-MOP.
